(19) United States
(12) Patent Application Publication
(10) Pub. No.: US 2016/0289778 A1
(43) Pub. Date: Oct. 6, 2016

(54) HCV GENOTYPING ALGORITHM

(71) Applicant: VELA OPERATIONS PTE. LTD., Singapore (SG)

(72) Inventors: Pramila Nuwantha ARIYARATNE, Singapore (SG); Charlie Wah Heng LEE, Singapore (SG)

(21) Appl. No.: 15/035,173

(22) PCT Filed: Oct. 31, 2014
(86) PCT No.: PCT/IB2014/065738

§ 371 Date: May 6, 2016

(30) Foreign Application Priority Data
Nov. 6, 2013 (GB) 1319580.5

(51) Int. Cl. C12Q 1/70; G06F 19/00; G06F 19/22

(52) U.S. Cl. CPC C12Q 1/707; G06F 19/22; G06F 19/345; C12Q 2600/106; C12Q 2600/158

(57) ABSTRACT

The present invention relates to methods for determining genotypes of pathogens present in a sample, e.g. a clinical sample, using next generation sequencing, in particular ion semiconductor sequencing. The present invention also relates to apparatus comprising computer units for carrying out the genotyping methods disclosed herein as well as to software products suitable for the execution of the methods disclosed herein.

The present invention relates to methods for determining genotypes identified in a sample, e.g. a clinical sample, using next generation sequencing, in particular ion semiconductor sequencing. The present invention also relates to devices comprising computer units for carrying out the genotyping methods disclosed herein as well as to software products suitable for the execution of the methods disclosed herein.

### BACKGROUND

A number of pathogens are associated with substantial individual suffering and important socio-economic consequences, such as healthcare costs, etc.. Frequently pathogens, such as bacteria and viruses exist as different strains, genotypes or subtypes. Individual strains, genotypes or subtypes may be more or less susceptible to available treatments, e.g. antibiotics or anti-viral drugs. In recent years, the development of antibiotic-resistant bacteria and the development of viruses that are not or less affected by known anti-viral drugs were a source of major concern. For example, the increasing number of Staphylococcus aureus strains, Mycobacterium tuberculosis strains, etc. that cannot be treated with antibiotics that were sufficiently effective in the past was matched by a higher number of fatalities and associated socio-economic consequences, e.g. days of hospitalization, overall increases of the days required to recover from infections, etc.

Furthermore, important viral pathogens for humans, for example HIV, HCV and HBV, deserve also higher attention due to the development of virus variants that are not sufficiently eliminated by currently known anti-viral drugs, e.g. gamma interferon, anti-retroviral drugs, etc.. These viruses have high rates of genetic mutation that may alter drug binding sites, thereby conferring partial or complete resistance towards known drugs. This results in higher titers of resistant pathogens in affected patients with consequences, both for the afflicted individual and the society.

An example for a virus that is socio-economically important and known for the development of mutations causing resistance towards some anti-viral drugs is hepatitis C virus (HCV). This virus is an example to illustrate the present invention.

HCV is related to the Flaviviridae RNA-containing virus family and causes an infectious process with the most frequent complication of liver cirrhosis and hepatocarcinoma (CDC Report N° 61; Younossi Z, Kallman J, Kincaid J. The effects of HCV infection and management on health-related quality of life; Hepatology. 2007 March; 45(3): 806-16). In 2005 an estimated number of more than 170 million people on the planet were afflicted by this disease (Robert-Koch-Institut: Epidemiologisches Bulletin. 46/2005), and the number of the affected is still rising.

Acute infection with HCV (20% of all acute hepatitis infections) frequently leads to chronic hepatitis (70% of all chronic hepatitis cases) and end-stage cirrhosis. It is estimated that up to 20% of HCV chronic carriers may develop cirrhosis over a time period of about 20 years and that of those with cirrhosis between 1 to 4%/year is at risk to develop liver carcinoma (Shiffman 1999; Lauer and Walker 2001).

An option to increase the life-span of HCV-caused end-stage liver disease is liver transplantation (30% of all liver transplantations world-wide are due to HCV-infection).

Further, when a liver-transplantation is not indicated or not feasible, a contemporary wide-spread trend in HCV treatment is the use of combination therapy comprising co-injection of megadoses of interferon with a cocktail containing both common antiviral preparations and one or two inhibitors of HCV replication (specific protease-helicase and/or RNA-polymerase inhibitors) (e.g., Toniutto P, Fabris C, Bitetto D, Fomasiere E, Rapetti R, Pirisi M. Valopicitabine dihydrochloride; a specific polymerase inhibitor of Hepatitis C virus. Curr Opin Investig Drugs. 2007 February; 8(2):150-8;). These cocktails increase the percentage of recovery, however inevitably leading to the formation of adaptive, inhibitor-resistant HCV mutants.

HCV has a high mutation rate, which is thought to help the virus in escaping its host's immune system. The high mutation rate is reflected in the presence of many distinct HCV genome sequences, known as quasispecies, within infected individuals (Bukh et al. 1995; Farci et al. 1997). Quasispecies result from the activity of the virally-encoded NS5B RNA-dependent RNA polymerase, which, due to its lack of proofreading function, is inherently a low-fidelity enzyme. The possible biological consequences of quasispecies include: (i) the development of escape mutants to humoral and cellular immunity leading to the establishment of a persistent infection; (ii) variable cell tropism (e.g., lymphotropic vs hepatotropic); (iii) vaccine failure, and (iv) rapid development of drug resistance.

The identification of a genotype and subtype of an HCV specimen is therefore of substantive importance for the purpose of detecting treatment response, evaluating duration and efficacy of antiviral therapy and establishing a route of virus propagation. Depending on the genotype of sub-type, patients are currently treated with interferon-based drugs which may be used for the treatment of infections with genotypes 2, 3, 5 and 6, whereas genotypes 1 and 4 are less responsive to interferon-based treatment. It is now well established that HCV exists as distinct genotypes among different HCV isolates with prevalence of each of the genotypes in specific geographical locations. At present, HCV variants are primarily classified into 6 genotypes, representing the 6 genetic groups defined by phylogenetic analysis of core/El and NS5B subgenomic sequences as well as of complete genome sequences. Within each genotype, HCV variants can be further divided into subtypes (Simmonds et al. 1994). An overview of the current unified system of nomenclature of HCV genotypes is given by Simmonds et al. (2005).

With the current standard FDA approved antiviral therapy of pegylated interferon a in combination with ribavirin, only approximately half of genotype-1 HCV infected individuals eligible for this treatment achieve a sustained virologic response (Manns et al. 2001). Because of severe side effects and other medical complications, up to 75% of the HCV patients are excluded from therapy today. Therefore, new anti-HCV drugs that are currently under development and in early stage clinical trials, are aimed at employing new targets such as the HCV internal ribosome entry site (IRES), the HCV NS3 serine protease and the HCV NS5B RNA-dependent RNA polymerase (see e.g. Tan et al. 2002; De Francesco et al. 2003).

However, the high mutation rate and variability of HCV are expected to favor the emergence of drug resistance, limiting the clinical usefulness of these inhibitors. In fact, drug resistance mutations have already been discovered during in vitro experiments and initial clinical trials. For example, HCV sub-genomic replicons have been used to study viral resistance to both nucleoside and non-nucleoside NS5B inhibitors as well as to NS3/4A protease inhibitors (Kukolj et al. 2005; Mo et al. 2005; reviewed in: Tomei et al. 2005). An understanding of HCV resistance mutants would further progress towards effective HCV treatments. In other words, evaluating the susceptibility of existing drug resistance mutants to different classes of antiviral agents is of utmost importance for the development of new drugs. In addition, exploring the effect of combination treatment on drug resistance may provide insight into future HCV treatment strategies. All this could enable appropriate therapeutic protocols to be rapidly developed and/or to be altered. As such, treating HCV-infected patients with an appropriate, i.e. active, drug would become much more feasible.

In order to perform such accurate drug resistance profiling across HCV genotypes, methods are needed to efficiently analyze HCV nucleic acid sequences. While HCV serves as an example of a pathogen that is evolving in response to therapeutic interventions similar concerns apply also to other pathogens that become more responsive to certain drugs, e.g. HIV, antibiotic-resistant bacteria (e.g. MRSA), etc.

The present invention addresses this need to determine reliably and accurately the genotype or subtype of pathogens, e.g. a virus such as HCV, and provides new methods and apparatus for determining virus genotypes using next generation sequencing.

Next generation sequencing is a method that is becoming more and more important in the diagnosing of diseases, e.g. infectious diseases such as viral infections. Next generation sequencing permits the determination of the sequence of nucleic acids, e.g. viral nucleic acids and provides important information for the physician when selecting of the correct treatment for an individual.

Next generation sequencing is based on the parallel sequencing of a huge number, e.g. thousands or millions, of sequences concurrently. Currently, various types of next generation sequencing methods are used, Roche's 454 pyrosequencing, Illumina sequencing, SOLiD sequencing and ion semiconductor sequencing being the most advanced sequencing methods. Several companies have introduced apparatus on the market that allow next generation sequencing methods to be performed in an automated fashion. Next generation sequencing is known to technical experts in the field and is principally based on the isolation of nucleic acids from a source, e.g. a clinical source, such as a clinical sample, generating short fragments of the nucleic acids with a size of several hundred to several thousand base pairs. These short fragments are cloned into a library and then individually sequences are introduced into separate reaction vessels where the sequencing reaction takes place. Different methods exist for sequencing nucleic acids, for example methods based on release of phosphate, release of fluorescence or the detection of positively charged hydrogen ions.

The present invention provides means to analyze the data obtained in the process of a next generation sequencing process. Quite often only short fragments of the desired genomic region are sequenced and have to be assembled into a contig covering essential all of the genomic region of interest.

Further, a clinical sample may contain different types of the desired target genomic region (for example a viral genomic region) representative for genotypes of the particular virus suspected to be present in a clinical sample, it is important to find out the exact nature of the genotype of the target gene (e.g. a viral gene) in order to find the correct treatment for the affected patient.

When a gene of a certain length (e.g. about 1000 base pairs) is sequenced using next generation sequencing methods such as ion semiconductor sequencing methods, a huge number of fragments covering the entire genomic region have to be reassembled after sequencing to form a contiguous sequence (a contig).

The present invention provides a surprisingly efficient method for assembling the information obtained in a huge number of individual sequencing reactions and comparing the obtained information with information previously gathered (e.g. in form of a gene database such as a database containing information on known HCV genotypes and subtypes) with very high efficiency.

### DEFINITIONS

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “detecting the presence” as used herein is to be understood in the meaning of “detecting the presence or absence”. As mentioned in the method as claimed in the present application, the sample to be analyzed is suspected to comprise a nucleic acid comprising a consensus nucleic acid sequence (which may also be designated as target sequence) indicative of the presence of a pathogen.

In the context of the present invention a “consensus nucleic acid sequence indicative of the presence of a pathogen” or “target sequence” designates a genomic region of a given pathogen that is specific for said pathogen. Amplification of the genomic region, e.g. using (RT-) PCR, and sequencing of the amplification product is, allows determining whether or not a given pathogen is present in a sample from which the amplified nucleic acid was obtained. The term “consensus” means that the genomic region allows specifically determining whether or not a nucleic acid sequence of a given pathogen is present in a sample, but takes into account that more than one nucleic acid sequence variants exists, i.e. more than one genotype, subtype or strain of said pathogen. For example, the NS5B genomic region of HCV allows identifying the presence of HCV in a sample. However, several genotypes and subtypes of this genomic region exist, i.e. while the genomic region comprises a consensus sequence indicative of all HCV genotypes these individual genotypes and subtypes have different nucleic acid sequences or variants of said nucleic sequences.

In the context of the present invention the term “nucleic acid” refers to a naturally occurring deoxyribonucleotide or ribonucleotide polymer in either single-or double-stranded form. The nucleic acid may particularly be double-stranded DNA and single-stranded RNA.

The term “sequence” as used herein refers to the sequential occurrence of the bases in a deoxyribonucleotide or ribonucleotide polymer, wherein a base found in a deoxyribonucleotide polymer is selected from the group consisting of A, T, G and C and a base found in a ribonucleotide polymer is selected from the group consisting of A, U, G and C. A sequence of bases in a deoxyribonucleotide polymer may thus e.g. be GGAAGCAAGCCT, whereas a sequence of bases in a ribonucleotide polymer may e.g. be GGAAUCGAU.

As used herein, the term “sample” refers to any biological sample from any human or veterinary subject that may be tested for the presence of a nucleic acid comprising a target sequence. The samples may include tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes. As listed above, samples may also be derived from a specific region in the body, e.g. the respiratory tract; samples from the respiratory tract include throat swabs, throat washings, nasal swabs, and specimens from the lower respiratory tract.

The sample may in particular be derived from a human or a veterinary subject. Accordingly, a “patient” may be a human or veterinary subject. If reference is made to a “clinical sample”, this indicates that the sample is from a patient suspected to be infected by a pathogen having a nucleic acid comprising a target sequence.

As used herein, the term “amplification” refers to enzyme-mediated procedures that are capable of producing billions of copies of nucleic acid target. Examples of enzyme-mediated target amplification procedures known in the art include PCR.

A “PCR reaction” has first been described for the amplification of DNA by Mullis et al. in U.S. Pat. No. 4,683,195 and Mullis in U.S. Pat. No. 4,683,202 and is well known to those of ordinary skill in the art. In the PCR technique, a sample of DNA is mixed in a solution with a molar excess of at least two oligonucleotide primers of that are prepared to be complementary to the 3′ end of each strand of the DNA duplex (see above, a forward and a reverse primer); a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand (in the above definition the non-sense strand) of the denatured DNA analyte and another is a reverse primer that will bind in the 3′ to 5′ direction to the 5′ end of the other strand (in the above definition the sense strand) of the denatured DNA analyte. The solution is heated to about 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR”.

The term “sequencing” is used herein in its common meaning in molecular biology. Thus, the exact sequential occurrence of bases in a nucleic acid sequence is determined.

The term “pathogen” as used herein is used in its broadest meaning. Thus, a pathogen may be any type of bacteria, archaeum, protozoum, fungus and virus. It is explicitly mentioned that viruses fall under the definition of a “microorganism” as used herein.

### EMBODIMENTS OF THE PRESENT INVENTION

In one aspect, the present invention relates to a method of (i) determining or detecting the presence or absence of a pathogen in a sample, and (ii) determining the genotype and/or subtype of a pathogen in a sample, said method comprising the following steps:
a. providing a sample suspected of containing a pathogen,
b. selecting a consensus sequence indicative of the presence said pathogen,
c. determining the nucleic acid sequences of the selected consensus sequence indicative of said pathogen using a software suitable for sequencing read assembly (e.g. MIRA),
d. providing a phylogenetic tree of a set of gene sequences with known genotypes constructed with a software using widely accepted multiple sequence alignment algorithms (e.g. MAFFT, CLUSTALW, MUSCLE) and phylogenetic tree construction algorithms (e.g. Maximum-likelihood, Nearest-neighbor, Maximum-parsimony),
e. aligning the obtained consensus nucleic acid sequences with the set of gene sequences with known genotypes indicative of said pathogen using a software suitable for sequence alignments (e.g. BLAST, TMAP, BWA),
f. determining the subset of gene sequences with high similarity to the obtained consensus nucleic acid sequences
g. determining the lowest common ancestors in the phylogenetic tree in step d) of the subset of gene sequences with high similarity to the obtained consensus nucleic acid sequences in step f),
h. based on the results obtained in step g), diagnosing the pathogen genotype/subtype present in the sample, and
i. optionally determining co-infections with at least two different genotypes or subtypes of the gene sequence indicative of said pathogen.

The samples suitable for analysis by the above methods can be any type of sample, in particular clinical samples as defined above. The provision of the sample means that a sample is removed from the organism that is suspected of containing (being infected by) a given pathogen. Further, the methods generally comprise a step wherein the nucleic acids in a sample are extracted and purified so that further analytical steps such as reverse transcription, PCR, etc. can be performed.

The above methods comprise a step wherein a consensus sequence indicative of the presence said pathogen is selected. This step relies on information on the genetic identity and variability of a given pathogen. For example, if HCV is the pathogen whose presence and genotype/subtype should be determined, databases provide information on those regions that allow both, specifically detecting HCV and determining the genotype/subtype of said HCV. It is clear that (RT-) PCR based amplification and detection methods require the design of primers specifically amplifying HCV genomic regions that permit specific amplification. Thus, the methods of the invention further comprise a step of designing PCR primers hybridizing specifically with genomic regions of HCV or their complements to allow specific amplification of the consensus genomic region, i.e. the genomic region of interest (target region). The length of the amplified genomic region is preferably in a range of about 100 to 1000 nucleotides, but longer fragments of genomic regions may also be amplified, if desired, e.g. regions of about 1100, 1200, 1300, 1400, 1500 nucleotides length, or longer. The selection of the targeted genomic region depends generally on the question, whether or not said genomic region is specific for a given pathogen, e.g. HCV, and whether or not further analysis of the amplification products permits determining the genotypes/subtypes of said pathogen. For example, if the amplification and analysis of a genomic region of about 800 nucleotides length is sufficient to specifically detect a pathogen, e.g. HCV, there is no need to obtain longer fragments, provided that this fragment (consensus genomic region) permits also determining the genotypes/subtypes of the respective pathogen.

The step of determining the nucleic acid sequence of the selected consensus sequence indicative of said pathogen is performed subsequent to the amplification of the nucleic acid sequence. The nucleic acid sequence is determined by sequencing, preferably next generation sequencing, most preferably ion semiconductor sequencing of the targeted genomic region of the pathogen. In the methods of the present invention, a larger number of amplification products representing the genomic region of interest is sequenced. The sequencing reads of all sequencing reactions are assembled to provide contiguous sequences (contigs). The assembly step is performed using a software that is freely accessible, which is known as MIRA assembler (i.e. software suitable for automatic assembly and editing of nucleotide sequences). Other software that may be used is known under the names Newbler, CLC Bio, etc. provided such software is suitable to direct such assembly of contigs. After assembly of the nucleotide sequences in contigs, the quality of the assembled contigs is checked in terms of coverage, length, wherein contigs that have at least 300 fold average coverage of the target nucleic acid sequence and that have a length of longer than 250 nucleotides, longer than 300 nucleotides, preferably contigs longer than 400 nucleotides, or still longer sequences are considered valid for the purpose of the present invention.

In the methods of the invention, a phylogenetic tree of a set of gene sequences with known genotypes indicative of said pathogen is generated using multiple sequence alignment algorithms and tree construction algorithms. Subsequently, the obtained nucleic acid sequences are aligned with the set of gene sequences with known genotypes using software suitable for sequence alignments, e.g. the TMAP software, which is a short read aligner specifically tuned for data from the Ion Torrent PGM. When the pathogen is HCV, the set of gene sequences with known genotypes can be gathered from the “Hepatitis C Virus (HCV) Database Project”, which was initially funded by the Division of Microbiology and Infectious Diseases of the National Institute of Allergies and Infectious Diseases (NIAID) is accessible to the public.

Due to the high mutation rates of viruses, it is unlikely that the obtained nucleic acid sequences will match exactly to a single known gene sequence in the database. Furthermore, many known gene sequences deposited in the database are highly similar to one another. Thus, for each nucleic acid sequence, a subset S of the gene sequences with known genotypes that matches most closely to the nucleic acid sequence based on the BLAST alignment scores is obtained. The genotype of the nucleic acid sequence is inferred from the genotype of A_{S}, the lowest common ancestor of the known gene sequences in S on the phylogenetic tree. The lowest common ancestor of a set of nodes N on a phylogenetic tree T is defined as the lowest node in T that has all nodes in N as descendants. Biologically, A_{S} represents the evolutionary parent of the known gene sequences in S. Using this approach, the genotyping accuracy improves because all genotype information from gene sequences that match closely to the nucleic acid sequence are used. Furthermore, recombination can be inferred if the gene sequences that match closely to a nucleic acid have different genotypes. Similarly, co-infections can be inferred if the nucleic acid sequences in the same sample have different genotypes.

An advantage of the methods of the present invention is that recombinations or unusual sequence variations can be detected by looking at the BLAST results. Further, it is possible also to detect this when top matches relate to sequences from different genotypes. It is possible, using the methods of the present invention to directly construct a phylogenetic tree using the contig sequence, wherein the sequence might be placed in some part of the tree that is closer to one or the other genotype. Generally, recombination is hard to detect in this case.

Another advantage associated with the methods of the present invention is the ability to detect co-infections, e.g. with at least two strains, genotypes or subtypes of a pathogen, e.g. two different genotypes or subtypes of HCV.

Subaspects of the above method relate to detection of a pathogen selected from the group of microorganisms comprising bacteria or viruses. As used herein, bacteria may preferably be selected from human pathogenic bacteria that have developed resistance to drugs such as antibiotics, e.g. methicillin resistant Staphylococcus aureus strains (MRSA), antibiotic-resistant Klebsiellae pneumoniae strains, antibiotic-resistant Mycobacterium tuberculosis strains, or toxin producing bacteria, such as EHEC (enterohemorrhagic E. coli strains).

Examples of viruses that are preferably detected or whose genotype or subtype is determined are selected from the group of human pathogenic viruses comprising HIV, HCV, HBV, norovirus, coronaviruses, papillomaviruses, adenoviruses, herpesviruses.

The methods of the present invention are preferably used for the detection of the presence or absence and the determination of the virus genotypes/subtypes of a virus selected from the group consisting of HCV, HIV, HBV, human herpesviruses, e.g. CMV, etc.

The methods of the present invention are particularly well-suited for the genotyping, or detecting of co-infections with more than one genotype or subtype when the pathogenic virus is HCV.

The methods of the present invention are particularly suitable for the genotyping, or detecting of co-infections with more than one genotype or subtype when the pathogenic virus is HCV, wherein the consensus sequence indicative (target sequence) of an infection is a fragment of the HCV NS5B genomic region comprising nucleotide positions 8614 and 9298.

The methods of the present invention are particularly suitable for the analysis of clinical samples, wherein the clinical samples is derived from a patient selected from the group consisting of:
a) patients suspected to be infected with HCV,
b) patients known to be infected with HCV, wherein the genotype and/or subtype of HCV is unknown,
c) patients previously untreated with an anti-viral drug;
d) patients already treated with an anti-viral drug,
e) patients not responding to an anti-viral drug,
f) patients infected with HCV that appears resistant to an anti-viral drug
g) patients infected with HCV and which are treated with an anti-viral drug in a clinical trial.

The methods of the present invention are preferably comprising a sequencing step, wherein the nucleotide sequence is determined by Next Generation Sequencing.

The methods of the present invention are particularly suitable, when the sequence is determined by sequencing fragments of said consensus nucleic acid (target) sequence and assembling the sequencing information into a contig.

The methods of the present invention are particularly adapted to assembling the nucleic acid sequence reads into a contig sequence using a software algorithm (e.g. the MIRA assembler, cf. http://www.chevreux.org/projects_main.html).

In the methods of the present invention, each contig is aligned with known gene sequences indicative of said pathogen using software suitable for sequence alignments, e.g. BLAST.

Also encompassed by the present invention is a software product comprising the software paths to carry out the steps of the methods referred to herein.

The present invention also relates to a method of selecting a treatment therapy comprising performing the methods of any of the preceding paragraphs and selecting the treatment based on the results of said method of determining the genotype/subtype of said pathogen. In a preferred embodiment of this aspect of the invention, the therapy for treating an infection by pathogen is specific for a virus selected from the group consisting of HCV, HIV, HBV, Herpesviruses, e.g. CMV. The most preferred embodiment of this aspect of the invention relates to the treatment of HCV infections. The consensus sequence indicative of an infection by HCV in this aspect of the invention is the HCV genomic region NS5B or a fragment thereof. Preferably, this fragment comprises nucleotide positions 8614 and 9298. However, it is explicitly contemplated to use other HCV genomic regions for the determination of suitable genotype- or subtype-specific therapeutic treatment.

The present invention also relates to a method of selecting a treatment therapy of HCV infected subjects comprising performing the methods of any of the preceding paragraphs. For example a treatment with e.g. Telaprevir plus Interferon and Ribavirin or Boceprivir plus Interferon and Ribavirin is suitable when the patient is infected with HCV genotype 1. For other genotypes, e.g. HCV2, 3 and 4, a treatment with Interferon plus Ribavirin may be selected.

The present invention also relates to an apparatus suitable to read software to perform any of the above methods for the detecting the presence and determination of the genotype of a pathogen, e.g. HCV. The apparatus is capable of executing the method steps provided for in the software.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXAMPLES

Blood samples obtained from human patients were used to extract nucleic acids that was later on subjected to preparatory steps for Next Generation Sequencing using an Ion Torrent semiconductor sequencing apparatus.

As consensus sequence indicative of the presence of HCV the NS5B region comprising nucleotide positions 8614 and 9298 was selected.

NGS sequencing was used to determine the nucleic acid sequences of the above selected consensus sequence indicative of HCV. MIRA (http://mira-assembler.sourceforge.net/docs/DefinitiveGuideToMIRA.html) suitable for sequencing read assembly was used to accomplish this step.

A phylogenetic tree of a set of gene sequences with known HCV genotypes was constructed with a software using a widely accepted multiple sequence alignment algorithms MAFFT (http://mbe.oxfordjournals.org/content/30/4/772) and phylogenetic tree construction algorithms (e.g. Maximum-likelihood http://code.google.com/p/phyml/).

The obtained consensus nucleic acid sequences were aligned with the set of gene sequences with known genotypes indicative of said pathogen using a software suitable for sequence alignments (e.g. TMAP from IonTorrent).

A subset of gene sequences with high similarity to the obtained consensus nucleic acid sequences was determined.

The lowest common ancestors in the phylogenetic tree in step d) of the subset of gene sequences with high similarity to the obtained consensus nucleic acid sequences in the preceding step was determined.

Based on the results obtained in the preceding step the pathogen genotype/subtype present in the sample were diagnosed.

1. A method of determining/detecting the presence or absence and/or the genotype and/or subtype of a pathogen in a sample, said method comprising the following steps:
a. providing a sample suspected of containing a pathogen,
b. selecting a consensus sequence indicative of the presence said pathogen,
c. determining the nucleic acid sequence of the selected consensus sequence indicative of said pathogen,
d. aligning the obtained nucleic acid sequence with known gene sequences indicative of said pathogen using (software suitable for sequence alignments) BLAST, and
e. determining the genotype or subtype of a pathogen based on the nucleic acid sequence of the selected consensus sequence of the genomic region indicative of said pathogen.

2. The method of claim 1, further comprising step f, wherein co-infections with at least two different genotypes or subtypes of the gene sequence indicative of said pathogen are determined.

3. The method of claim 1, wherein the pathogen is a virus selected from the group consisting of HCV, HIV, HBV, or a herpesvirus.

4. The method of claim 1, wherein the virus is HCV.

5. The method of claim 1, wherein the consensus sequence indicative of an infection is HCV a fragment of HCV NS5B genomic region comprising nucleotide positions 8614 to 9298.

6. The method of claim 1, wherein the sample is a clinical sample.

7. The method of claim 1, wherein the clinical sample is derived from a patient selected from the group consisting of:
a) patients suspected to be infected with HCV,
b) patients known to be infected with HCV, wherein the genotype and/or subtype of HCV is unknown,
c) patients previously untreated with an anti-viral drug;
d) patients already treated with an anti-viral drug,
e) patients not responding to an anti-viral drug,
f) patients infected with HCV that appears resistant to an anti-viral drug, etc.

8. The method of claim 1, wherein the sequence is determined by amplification of a part of the genomic region of NS5B and determining the nucleotide sequence.

9. The method of claim 1, wherein the nucleotide sequence is determined by Next Generation Sequencing.

10. The method of claim 1, wherein the sequence is determined by sequencing said consensus sequence and assembling the sequencing information into a contig.

11. The method of claim 1, wherein said the sequencing information is assembled into a contig sequence using a software algorithm.

12. The method of claim 1, wherein each contig is aligned with gene sequences indicative of said pathogen using software suitable for sequence alignments, preferably BLAST.

13. A software product comprising the software paths to carry out the steps of the method of claim 1.

14. A method of selecting a treatment therapy comprising performing the method of claim 1, further comprising the step of selecting the treatment based on the results of said method of determining the genotype/subtype of said pathogen.

15. The method according to claim 1, wherein the pathogen is a virus selected from the group consisting of HCV, HIV, HBV.

16. The method according to claim 14, wherein the virus is HCV.

17. The method according to claim 14, wherein the consensus sequence indicative of an infection with is the HCV genomic region NS5B 8614 to 9298.

18. The method according to claim 14, wherein the sample is a clinical sample.

19. The method according to claim 14, wherein the clinical sample is derived from a patient selected from the group consisting of
a. patients suspected to be infected with HCV,
b. patients known to be infected with HCV, wherein the genotype and/or subtype of HCV is unknown,
c. patients previously untreated with an anti-viral drug;
d. patients already treated with an anti-viral drug,
e. patients not responding to an anti-viral drug,
f. patients infected with HCV that are resistant to an anti-viral drug,
g. patients infected with HCV taking part in a clinical trial for an antiviral drug.

20. The method according to claim 14, wherein the sequence is determined by Next Generation Sequencing.

21. The method according to claim 14, wherein the sequence is determined by sequencing fragments of said consensus sequence and assembling the sequencing information into a contig.

22. The method according to claim 14, wherein said the sequencing information is assembled into a contig sequence using a software algorithm.

23. The method according to claim 14, wherein each contig is aligned with known gene sequences indicative of said pathogen using software suitable for sequence alignments, preferably BLAST.

24. The method according to claim 14, wherein a specific treatment with anti-viral drugs is selected depending on the HCV genotype.

25. A software product comprising the software paths to carry out the steps of the method of claim 14.

26. An apparatus capable of reading and executing the method steps defined in software according to claim 25.

27. The apparatus according to claim 26, wherein said apparatus is capable of executing the method steps provided for in the software according to claim
